(19) United States
(12) Patent Application Publication
(10) Pub. No.: US 2026/0130624 A1
(43) Pub. Date: May 14, 2026

(54) DRIVER STATE DETERMINATION DEVICE, DRIVER STATE DETERMINATION METHOD, AND STORAGE MEDIUM

(71) Applicant: HONDA MOTOR CO., LTD., Tokyo (JP)

(72) Inventors: Kazuma Hamada, Tokyo (JP); Hirotaka Katayama, Tokyo (JP); Yuichi Masukake, Wako-shi (JP)

(21) Appl. No.: 19/381,027

(22) Filed: Nov. 6, 2025

(30) Foreign Application Priority Data
Nov. 8, 2024 (JP) 2024-196033

(51) Int. Cl. A61B 5/00; B60Q 9/00; G07C 5/04

(52) U.S. Cl. CPC A61B 5/4806; B60Q 9/00; A61B 2503/22; G07C 5/04

(57) ABSTRACT

A driver state determination device includes a determiner configured to determine a state of a driver of a mobile object, and a notifier configured to issue a notification to the driver on the basis of a determination result of the determiner, in which the notifier issues a first notification when the determiner determines that a state of the driver is a first state, and issues a second notification different from the first notification when the determiner determines that the state of the driver is a second state different from the first state.

### CROSS-REFERENCE TO RELATED APPLICATION

Priority is claimed on Japanese Patent Application No. 2024-196033, filed November 08, 2024, the content of which is incorporated herein by reference.

### BACKGROUND

### FIELD OF THE INVENTION

The present invention relates to a driver state determination device, a driver state determination method, and a storage medium.

### DESCRIPTION OF RELATED ART

In recent years, efforts to provide access to sustainable transportation systems that take into consideration vulnerable traffic participants have been gaining momentum. To realize this goal, efforts are being focused on research and development of preventive safety technologies to further improve traffic safety and convenience. In this regard, conventionally, technologies that issue a drowsy driving warning based on a drowsiness level that match a driver's perception are known (for example, Japanese Unexamined Patent Application, First Publication No. 2019-113925).

### SUMMARY

However, in conventional preventive safety technologies, when a state of a driver is determined based on a drowsiness level, it can sometimes be difficult to determine whether the driver is drowsy or simply has his or her eyes closed, similar to inattentive driving. For this reason, there is an issue that the state of the driver may not be appropriately determined.

To solve the problems described above, one objective of this application is to provide a driver state determination device, a driver state determination method, and a storage medium that can more accurately determine a state of a driver. This, in turn, contributes to the development of sustainable transportation systems.

The driver state determination device, the driver state determination method, and the storage medium of the present invention have adopted the following configuration.

(1): A driver state determination device according to one aspect of the present invention includes a determiner configured to determine a state of a driver of a mobile object, and a notifier configured to issue a notification to the driver on the basis of a determination result of the determiner, in which the notifier issues a first notification when the determiner determines that a state of the driver is a first state, and issues a second notification different from the first notification when the determiner determines that the state of the driver is a second state different from the first state.

(2): In the aspect of (1) described above, the first state is inattentive driving and the second state is drowsy driving.

(3): In the aspect of (2) described above, when the determiner determines that the state of the driver is the second state, the notifier issues a notification corresponding to the drowsy driving, and then issues a notification urging the driver to take a rest.

(4): In the aspect of (1) described above, when the driver has his or her eyes continuously closed and the driver has been driving the mobile object for a predetermined period of time or longer, the determiner determines that the state of the driver is the second state.

(5): In the aspect of (1) described above, the determiner determines that the state of the driver is the first state when the driver has his or her eyes continuously closed and a speed at which the driver closes his or her eyes is equal to or greater than a predetermined speed, and determines that the state of the driver is the second state when the speed at which the driver closes his or her eyes is less than the predetermined speed.

(6): In the aspect of (1) described above, the determiner determines that the state of the driver is the first state when the driver has his or her eyes continuously closed and the speed at which the driver closes his or her eyes is equal to or greater than a predetermined speed and the driver's mouth is closed, and determines that the state of the driver is the second state when the speed at which the driver closes his or her eyes is less than the predetermined speed and the driver's mouth is open.

(7): In the aspect of (1) described above, the determiner determines that the state of the driver is the second state when the driver has his or her eyes continuously closed and a behavior of the mobile object is not stable.

(8): In the aspect of (1) described above, the determiner determines that the state of the driver is the first state when the driver has continuously closed his or her eyes, a duration of driving the mobile object is less than a predetermined period of time, a drowsiness level of the driver is less than a predetermined value, and the behavior of the mobile object is determined to be stable.

(9): A driver state determination method according to another aspect of the present invention includes, by a computer, determining a state of a driver of a mobile object, issuing a notification to the driver on the basis of a result of the determination, issuing a first notification when a state of the driver is determined to be a first state, and issuing a second notification different from the first notification when the state of the driver is determined to be a second state different from the first state.

(10): A storage medium according to still another aspect of the present invention is a computer-readable non-transitory storage medium that has stored a program causing a computer to execute determining a state of a driver of a mobile object, issuing a notification to the driver on the basis of a result of the determination, issuing a first notification when a state of the driver is determined to be a first state, and issuing a second notification different from the first notification when the state of the driver is determined to be a second state different from the first state.

According to the aspects of (1) to (10) described above, it is possible to more appropriately determine a state of a driver.

### BRIEF DESCRIPTION OF THE DRAWINGS

FIG. 1 is a configuration diagram of a vehicle system including a driver state determination device according to an embodiment.

FIG. 2 is a diagram which schematically shows content of processing performed by a state recognizer.

FIG. 3 is a diagram (part 1) for describing an example of a method for recognizing an eye-opening rate.

FIG. 4 is a diagram (part 2) for describing an example of the method for recognizing the eye-opening rate.

FIG. 5 is a flowchart which shows an example of processing executed by a driving assistance device according to an embodiment.

### DESCRIPTION OF EMBODIMENTS

Embodiments of a driver state determination device, a driver state determination method, and a storage medium of the present invention will be described below with reference to the drawings. An example of a mobile object in which the driver state determination device is applied will be described below. A vehicle will be used as an example of a mobile object. In addition to vehicles, mobile objects may also include, for example, ships capable of moving on land (roads) such as hovercrafts, aircraft capable of traveling on roads, stand-up vehicles with power units, and micromobility vehicles such as electric kick scooters.

### [Overall configuration]

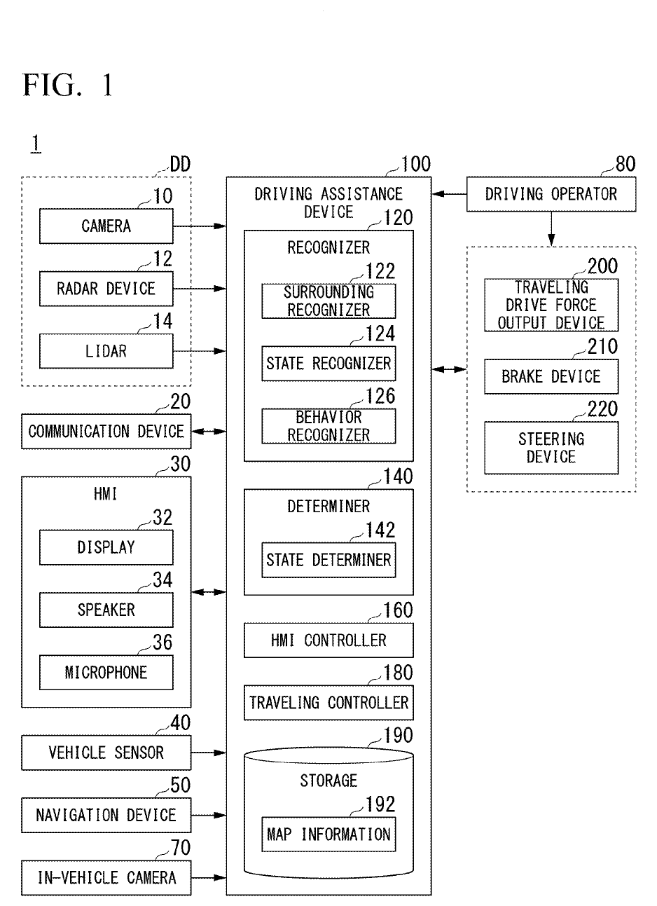

FIG. 1 is a configuration diagram of a vehicle system 1 including a driver state determination device according to an embodiment. A vehicle (hereinafter referred to as a vehicle M) on which the vehicle system 1 is mounted may be, for example, a two-wheeled, three-wheeled, or four-wheeled vehicle or a micromobility vehicle. A power source thereof may be an internal combustion engine such as a diesel engine or a gasoline engine, an electric motor, or a combination of these. The electric motor operates using power generated by a generator connected to the internal combustion engine or power discharged from a battery (storage battery) such as a secondary battery or a fuel cell.

The vehicle system 1 includes, for example, a camera 10, a radar device 12, a light detection and ranging (LIDAR) 14, a communication device 20, a human machine interface (HMI) 30, vehicle sensors 40, a navigation device 50, an in-vehicle camera 70, a driving operator 80, a driving assistance device 100, a traveling drive force output device 200, a brake device 210, and a steering device 220. These devices and apparatuses are connected to each other via multiplexed communication lines such as controller area network (CAN) communication lines, serial communication lines, a wireless communication network, or the like. Constituents shown in FIG. 1 are merely examples; some of the constituents may be omitted, or additional constituent may also be added. The camera 10, the radar device 12, and the LIDAR 14 are examples of a “detection device DD.” The driving assistance device 100 is an example of a “driver state determination device.”

The camera 10 is a digital camera that uses a solid-state imaging element such as a charge coupled device (CCD) or complementary metal oxide semiconductor (CMOS). The camera 10 is attached to any place on the vehicle M in which the vehicle system 1 is installed. To capture images of the front, the camera 10 is attached to a top of the front windshield, a back of the rearview mirror, a front of the vehicle body, or the like. To capture images of the rear, the camera 10 is attached to a top of the rear windshield, the back door, or the like. To capture images of the side, the camera 10 is attached to the left and right door mirrors, or the like. The camera 10, for example, periodically and repeatedly captures images of surroundings of the vehicle M. The camera 10 may be a stereo camera.

The radar device 12 emits radio waves (radar) such as millimeter waves to the surroundings of the vehicle M and detects radio waves reflected by a surrounding object (reflected waves) to determine at least a position (distance and orientation) of the object. The radar device 12 is attached to any place on the vehicle M. The radar device 12 may detect a position and a speed of an object using a frequency modulated continuous wave (FM-CW) method.

The LIDAR 14 emits light to the surroundings of the vehicle M and measures scattered light. The LIDAR 14 detects a distance to an object based on time between light emission and light reception. The emitted light may be, for example, pulsed laser light. The LIDAR 14 is attached to any place on the vehicle M.

The communication device 20 uses networks such as a cellular network, a Wi-Fi network, Bluetooth (registered trademark), dedicated short range communication (DSRC), a local area network (LAN), a wide area network (WAN), and the Internet to communicate with, for example, other vehicles in the surroundings of the vehicle M, terminal devices of users using the vehicle M, or various server devices.

The HMI 30 outputs various types of information to an occupant of the vehicle M (including the driver) and receives input operations from the occupant. The HMI 30 includes, for example, a display 32, a speaker 34, and a microphone 36. The display 32 is, for example, a liquid crystal display (LCD) or an organic electro luminescence (EL) display device. The display 32 displays various images (including videos) in the embodiment. The display 32 may be integrated with an input as a touch panel. The speaker 34 outputs a predetermined voice (for example, a voice alarm or a voice message). The microphone 36 receives voices from the occupant, including the driver. The HMI 30 may also include a buzzer, a touch panel, switches, keys, and the like. The switches may include switches that execute or terminate predetermined driving control that can be executed by a traveling controller which will be described below, and switches that approve (permit) or reject driving control recommendations (proposals) from a system (the vehicle system 1). The switches may also include a switch for performing a direction indication operation (blinker switch), and the like.

The vehicle sensor 40 includes a vehicle speed sensor that detects a speed of the vehicle M, an acceleration sensor that detects acceleration, and a yaw rate sensor that detects a yaw rate (for example, a rotational angular speed around a vertical axis passing through a center of gravity of the vehicle M). The vehicle sensor 40 may also include a lateral acceleration sensor (a lateral G sensor) that detects a lateral acceleration (lateral G) of the vehicle M, a steering angle sensor that detects a steering angle of the vehicle M (which may be an angle of the steering wheel or an operating angle of the steering wheel), a steering angular speed sensor that detects a steering angular speed, and an orientation sensor that detects a direction of the vehicle M. The vehicle sensor 40 may include, for example, a sensor that detects an operating state of the vehicle M (whether the vehicle system 1 is operating) or a distance traveled by the vehicle M.

The vehicle sensor 40 may include a position sensor that detects a position of the vehicle M. The position sensor is, for example, a sensor that acquires position information (longitude and latitude information) from a global positioning system (GPS) device. The position sensor may also be, for example, a sensor that acquires position information using a global navigation satellite system (GNSS) receiver in the navigation device 50. The vehicle sensor 40 may derive a speed of the vehicle M from a difference in position information (that is, a distance) over a predetermined period of time from the position sensor. Results detected by the vehicle sensor 40 are output to the driving assistance device 100.

The navigation device 50 includes, for example, a GNSS receiver, a navigation HMI, and a route determiner. The navigation device 50 may hold map information in a storage device such as a hard disk drive (HDD) or flash memory, or may acquire map information 192 stored in a storage 190 (described below). The GNSS receiver identifies the position of the vehicle M on the basis of signals received from GNSS satellites. The position of the vehicle M may be identified or supplemented by an inertial navigation system (INS) that uses an output of the vehicle sensor 40. The navigation HMI includes a display device, a speaker, a touch panel, a key, and the like. The GNSS receiver may be provided in the vehicle sensor 40. The navigation HMI may share some or all of the HMI 30 described above. The route determiner determines, for example, a route (hereinafter, a “map route”) based on the position of the vehicle M identified by a GNSS receiver (or any other input position) to a destination input by an occupant using a navigation HMI, by referring to, for example, map information 192. The navigation device 50 provides route guidance using the navigation HMI on the basis of the determined map route. The navigation device 50 may transmit a current position and the destination to a navigation server via the communication device 20 and acquire a route equivalent to the map route from the navigation server.

Here, the map information 192 is information that represents road shapes using, for example, links indicating roads (examples of moving paths) and nodes connected by the links. The map information 192 may also include point-of-interest (POI) information and the like. The map information 192 includes, for example, the number of lanes (the number of moving paths), types and shapes of road dividing lines, information on centers of lanes, and information on road boundaries. The map information 192 may also include information on whether road boundaries are boundaries (physical boundaries) that include structures that vehicles cannot pass through (including cross or come into contact with). Examples of physical boundaries include guardrails, curbs, medians, and fences. The map information 192 may also include road shape information, traffic regulation information, address information (address and postal code), facility information, parking information, and telephone number information. Road shape information includes, for example, road curvature (which may be referred to as curvature radius. The same applies below), road width, road surface gradient, branching points, merging points, intersections, and T-junctions. The map information 192 may be updated as needed by the communication device 20 communicating with an external device.

The in-vehicle camera 70 is a digital camera that uses a solid-state imaging element such as a CCD or CMOS. The in-vehicle camera 70 is attached to any place on the vehicle M in a position and direction that allows it to capture an image of a head of a driver seated in a driver's seat of the vehicle M from the front. For example, the in-vehicle camera 70 is attached near a display device (for example, at the top or bottom) provided in a center of an instrument panel of the vehicle M. The in-vehicle camera 70 may capture images of an interior of the vehicle by emitting infrared light toward the interior. The in-vehicle camera 70 captures images of the interior of the vehicle M in an area that includes an occupant (passenger) seated in a passenger seat of the vehicle M.

The driving operator 80 includes, for example, a steering wheel, an accelerator pedal, and a brake pedal. The driving operator 80 may also include a shift lever, differential steering, a joystick, or other operators. Each operator of the driving operator 80 is equipped with, for example, an operation detector that detects an amount of operation of the operator by the driver or presence or absence of an operation. The operation detector detects, for example, a steering wheel angle and a steering torque (for example, an amount of steering (steering input torque) by a driving operation of the driver), a rate of change in steering torque, amounts of depression of the accelerator pedal and the brake pedal, and the like. The operation detector then outputs a result of the detection to the driving assistance device 100 or one or both of the traveling drive force output device 200, the brake device 210, and the steering device 220. The driving operator 80 may include a direction indication operator (for example, a turn signal lever (turn lever), a turn signal switch). When the direction indication operator is operated, a turn signal of the vehicle M associated with an operation content flashes, and the operation content (including, for example, a result of detecting an operation that has been performed by the driver) is output to the driving assistance device 100.

The driving assistance device 100 executes various types of control to assist driving of a driver of the vehicle M. The driving assistance device 100 includes, for example, a recognizer 120, a determiner 140, an HMI controller 160, a traveling controller 180, and a storage 190. The recognizer 120, the determiner 140, the HMI controller 160, and the traveling controller 180 are each realized by a hardware processor, such as a central processing unit (CPU), executing a program (software). Some or all of these components may be realized by hardware (circuit; including circuitry), such as large scale integration (LSI), an application specific integrated circuit (ASIC), a field-programmable gate array (FPGA), a graphics processing unit (GPU), or a system on chip (SOC), or may be realized by a combination of software and hardware. The program described above may be stored in advance in a storage device (a storage device with a non-transitory storage medium) such as an HDD or flash memory of the driving assistance device 100, or may be stored in a removable storage medium such as a DVD, CD-ROM, or memory card and installed in a storage device of the driving assistance device 100 by mounting a storage medium (non-transitory storage medium) in a drive device, a card slot, or the like. The traveling controller 180 is an example of a “moving controller.” The HMI 30 and the HMI controller 160 are examples of a “notifier.”

The storage 190 may be realized by the various storage devices described above, or by an electrically erasable programmable read-only memory (EEPROM), a read-only memory (ROM), or a random access memory (RAM). The storage 190 stores, for example, map information 192, various types of information in the embodiment, programs, and the like. The storage 190 may store various types of setting information used in processing of the present embodiment.

The recognizer 120 includes, for example, a surrounding recognizer 122, a state recognizer 124, and a behavior recognizer 126. The surrounding recognizer 122 recognizes surrounding conditions of the vehicle M, for example, on the basis of results of detection by the detection device DD (information input from the camera 10, the radar device 12, and the LIDAR 14). For example, the surrounding recognizer 122 performs sensor fusion processing on results of detection by some or all of the camera 10, the radar device 12, and the LIDAR 14, and recognizes a state of an object present in the surroundings of the vehicle M (within a predetermined distance from the vehicle M), such as a position (relative position), a size, a speed (relative speed), and acceleration. Objects recognized by the surrounding recognizer 122 may include, for example, physical boundaries that divide roads (moving paths), as well as traffic participants (examples of obstacles) such as other vehicles, pedestrians, and bicycles. A position of an object is recognized as, for example, a position on an absolute coordinate system with a representative point of the vehicle M (such as a center of gravity or a center of a drive shaft) as the origin, and is used for control. The position of an object may be represented by a representative point such as a center of gravity or a corner of the object, or by an expressed area. For example, when the object is another vehicle, the “state” of the object may include acceleration or jerk of the other vehicle, or a “behavioral state” (for example, whether the other vehicle is changing lanes or about to change lanes).

The surrounding recognizer 122 may also recognize, for example, stop lines, red lights, toll booths, other road phenomena, road signs, and markings painted on a road (for example, speed limits). The surrounding recognizer 122 may recognize a curvature of a lane on which the vehicle M is traveling (traveling road) on the basis of the detection results of the detection device DD or the map information 192. The surrounding recognizer 122 may also recognize conditions of a road surface (for example, whether the road surface is slippery, such as frozen) on the basis of the detection results of the detection device DD.

The surrounding recognizer 122 recognizes, for example, a lane on which the vehicle M is traveling (traveling lane) and other surrounding lanes (for example, adjacent lanes). For example, the surrounding recognizer 122 recognizes road dividing lines from images captured by the camera 10 and recognizes the traveling lane and other lanes on the basis of a positional relationship of the recognized road dividing lines as seen from the vehicle M. The surrounding recognizer 122 may recognize the lane on which the vehicle M is traveling and other lanes by referring to the map information 192 on the basis of position information of the vehicle M obtained from the vehicle sensor 40, and the like.

The state recognizer 124 recognizes a state of the occupant of the vehicle M using images captured by the in-vehicle camera 70. For example, the state recognizer 124 performs known image analysis processing on the images captured by the in-vehicle camera 70 and recognizes an eye open or close state of the driver of the vehicle M on the basis of a result of the analysis. The state recognizer 124 may also recognize a mouth open or close state of the driver. The state recognizer 124 may also perform known audio analysis processing on a voice emitted by the driver acquired by the microphone 36 and recognize content of the voice on the basis of a result of the analysis, or may recognize a line of sight or a facial direction of the driver. Functions of the state recognizer 124 will be described in detail below.

The behavior recognizer 126 recognizes a behavior of the vehicle M on the basis of a detection result by the vehicle sensor 40, a detection result by an operation detector of the driving operator 80, and control content executed by the traveling controller 180. The behavior of the vehicle M includes behavior resulting from manual driving by the driver and behavior resulting from driving control executed by the traveling controller 180. The behavior recognizer 126 may recognize whether the behavior of the vehicle M is stable (or unstable).

For example, the behavior recognizer 126 may recognize a lateral position of the vehicle M relative to the traveling lane (a position in a lane width direction) and a posture (direction) of the vehicle M relative to an extension direction of the traveling lane on the basis of the positional relationship of the vehicle M relative to the traveling lane. The behavior recognizer 126 may recognize a deviation of a reference point of the vehicle M from a center of the lane and an angle which the reference point of the vehicle M forms against a line connecting centers of the lane in a traveling direction of the vehicle M as relative position and posture of the vehicle M relative to the traveling lane. Alternatively, the behavior recognizer 126 may recognize a position of a reference point of the vehicle M relative to one of side edges of the traveling lane (a road dividing line or road boundary) or the like as the relative position (lateral position) of the vehicle M relative to the traveling lane. The behavior recognizer 126 may also recognize the lateral behavior of the vehicle M (for example, whether the vehicle is moving laterally for a predetermined distance or more in a predetermined period of time) based on the lateral position of the vehicle M or an amount of change in direction (yaw rate) of the vehicle M.

The behavior recognizer 126 may detect the behavior of the vehicle M based on an amount of steering wheel operation (for example, steering angle, steering torque, steering torque change rate), an amount of depressing an accelerator pedal and a brake pedal, and the like, obtained by the operation detector when the vehicle M is being manually driven, and may also recognize whether the behavior is stable based on the amount of change in behavior over a predetermined period of time.

The behavior recognizer 126 may recognize that the behavior of the vehicle M is stable when an amount of change in the lateral position or direction of the vehicle M over a predetermined period of time is less than a threshold value, and may recognize that the behavior is not stable when the amount of change is equal to or greater than the threshold value. The behavior recognizer 126 may recognize that the behavior of the vehicle M is stable when an amount of change in acceleration or deceleration of the vehicle M over a predetermined period of time is less than a threshold value, and may recognize that the behavior is not stable when the amount of change is equal to or greater than the threshold value.

The behavior recognizer 126 may recognize a behavior of the vehicle M on the basis of content of driving control executed by the traveling controller 180. Driving control is, for example, control to cause the vehicle M to travel by controlling at least one of steering and a speed of the vehicle M, regardless of a driving operation by the driver or by receiving some instructions. The driving control may include, for example, an adaptive cruise control system (ACC), a lane keeping assistance system (LKAS), auto lane changing (ALC), and the like. The driving control may include control to stop the vehicle M at a safe position, such as a road shoulder, on the basis of a result of the determination by the determiner 140, and control of the steering and speed to avoid contact between the vehicle M and an obstacle recognized by the surrounding recognizer 122. The driving control may include, for example, a vehicle stability assist (VSA). The VSA is driving control for automatically stabilizing the behavior of the vehicle M when skidding or the like of the vehicle M occurs. Examples of the VSA include safety devices such as an anti-lock braking system (ABS) that reduces wheel skidding due to wheel lock during sudden deceleration or deceleration on low-friction roads, a traction control system (TCS) that prevents wheel spin during starting and acceleration, and a skidding suppression device, a system that comprehensively controls these safety devices, or the like. For example, the behavior recognizer 126 may recognize that the behavior of the vehicle M is not stable when the traveling controller 180 is executing the VSA, and may recognize that the behavior of the vehicle M is stable after the execution of the VSA has terminated.

The behavior recognizer 126 may recognize a duration of driving by the driver on the basis of results of detection by the vehicle sensor 40, and the like. The duration of driving may be time from when the vehicle system 1 is activated (for example, when an ignition switch of the vehicle M is turned on) to when the vehicle system 1 is terminated (for example, when the ignition switch is turned off), or may be time while the vehicle M is moving (excluding time when the vehicle M is stopped). The behavior recognizer 126 may also recognize a traveling distance (driving mileage) of the vehicle M by the driving of the driver. The traveling distance may be, for example, a distance traveled from when the vehicle system 1 is activated to when it is terminated.

The determiner 140 includes, for example, a state determiner 142. The state determiner 142 determines a state of the driver of the vehicle M. For example, the state determiner 142 determines whether the driver is in a predetermined state on the basis of a result of the recognition by the state recognizer 124. The predetermined state may be at least a first state or a second state, and may also be another state. The first state is, for example, inattentive driving of the driver. The second state is, for example, drowsy driving of the driver. A function of the state determiner 142 will be described in detail below.

The HMI controller 160 notifies the occupant (including the driver) of predetermined information via the HMI 30 and receives information input via the HMI 30. The predetermined information includes, for example, information related to traveling of the vehicle M, such as information on a state of the vehicle M and information on driving control. Information on the state of the vehicle M includes, for example, a speed, an engine speed, and a shift position of the vehicle M. Information on driving control includes, for example, information regarding whether driving control is being performed by the traveling controller 180 or an execution status of driving control, information on driving control recommendations (proposals) from the system, and information on a notification (warning, and the like) to the driver. The predetermined information may include information on the surrounding conditions recognized by the detection device DD. The predetermined information may also include information which is not related to the traveling of the vehicle M, such as television programs, content (for example, videos) stored on a storage medium such as a DVD, or the like. The predetermined information may also include, for example, information on a current position or a destination of the vehicle M, and a remaining fuel level of the vehicle M. The HMI controller 160 may output the information received by the HMI 30 to the communication device 20, navigation device 50, recognizer 120, determiner 140, traveling controller 180, and the like.

The HMI controller 160 may generate inquiry information or recommendation information for the occupant, a result of recognition by the recognizer 120, a result of determination by the determiner 140, notification information (first and second notifications described below), and the like, and output the generated information to the HMI 30. The generated information includes images and voices (including voice alarms). The HMI controller 160 may transmit various types of information output by the HMI 30 to a terminal device used by the occupant of the vehicle M via the communication device 20.

The traveling controller 180 controls the traveling of the vehicle M (movement of a mobile object). For example, the traveling controller 180 executes driving control for the vehicle M on the basis of the result of recognition by the recognizer 120 and the result of determination by the determiner 140. Driving control may be executed in response to an execution instruction from the driver via the HMI 30, or may be executed independently of an instruction from the driver, on the basis of the result of recognition by the recognizer 120. When driving control is executed, the traveling controller 180 generates a future target trajectory of the vehicle M according to content of the driving control on the basis of the result of recognition by the recognizer 120, and controls at least one of the steering and speed of the vehicle M so that the vehicle M travels along the generated target trajectory.

For example, the traveling controller 180 performs, for example, driving control such as ACC, LKAS, ALC, and VSA. When a monitoring direction of the driver does not improve within a predetermined period of time after the HMI controller 160 outputs notification information indicating that the monitoring direction of the driver is not appropriate, the traveling controller 180 performs control such as stopping the vehicle M in a safe position such as a shoulder of a road, or avoiding contact between the vehicle M and an obstacle.

The traveling drive force output device 200 outputs traveling drive force (torque) for traveling of the vehicle to drive wheels. The traveling drive force output device 200 includes, for example, a combination of an internal combustion engine, an electric motor, and a transmission, and an electronic control unit (ECU) that controls these. The ECU controls the constituents described above according to information input from the traveling controller 180 or information input from an accelerator pedal of the driving operator 80.

The brake device 210 includes, for example, a brake caliper, a cylinder that transmits hydraulic pressure to the brake caliper, an electric motor that generates hydraulic pressure in the cylinder, and a brake ECU. The brake ECU controls the electric motor according to the information input from the traveling controller 180 or the information input from the brake pedal of the driving operator 80, so that a brake torque corresponding to a braking operation is output to each wheel. The brake device 210 may include a backup mechanism that transmits hydraulic pressure generated by a brake pedal operation to the cylinder via a master cylinder. The brake device 210 is not limited to the constituent described above, and may also be an electronically controlled hydraulic brake device that controls an actuator according to the information input from the traveling controller 180 and transmits hydraulic pressure from the master cylinder to the cylinder.

The steering device 220 includes, for example, a steering ECU and an electric motor. The electric motor, for example, applies force to a rack-and-pinion mechanism to change a direction of the steered wheels. The steering ECU drives the electric motor to change a direction of the steering wheel in accordance with the information input from the traveling controller 180 or information input from the steering wheel of the driving operator 80.

### [State recognizer]

Next, the functions of the state recognizer 124 will be described in detail. The state recognizer 124 performs image analysis, such as edge extraction, on the images captured by the in-vehicle camera 70, and uses facial feature information obtained as a result of this analysis to recognize an eye-opening rate and a mouth-opening rate as examples of eye and mouth opening states of the driver.

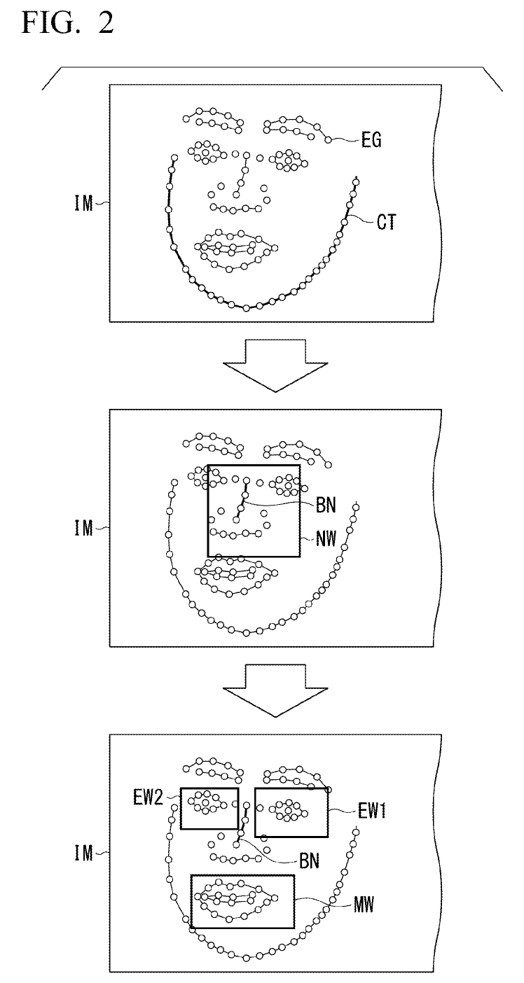

FIG. 2 is a diagram which schematically shows content of processing performed by the state recognizer 124. In FIG. 2, IM represents an image captured by the in-vehicle camera 70 with an edge EG superimposed on it. FIG. 2 focuses solely on a driver seated in the driver's seat. As shown in an upper diagram of FIG. 2, the state recognizer 124 first extracts a facial contour CT of the driver by fitting an elliptical, egg-shaped, or other model to the edge EG. Next, as shown in a middle diagram of FIG. 2, the state recognizer 124 sets a nose detection window NM based on the facial contour CT and detects a position and a shape of a bridge of the nose BN, which is a part within the nose detection window NM where edges are likely to be clearly extracted. Next, as shown in a lower diagram of FIG. 2, the state recognizer 124 sets eye detection windows EW1 and EW2 of predetermined sizes on right and left sides of the bridge of the nose BN, where eyes (right eye and left eye) of the driver are predicted to be present, based on the position and shape of the bridge of the nose BN, and the state recognizer 124 detects at least a portion of the eyes in each of the eye detection windows EW1 and EW2.

The state recognizer 124 may set a mouth detection window MW of a predetermined size below the bridge of the nose BN, where the driver’s mouth is predicted to be present, based on the contour CT and the position and shape of the bridge of the nose BN, and detect at least a portion of the driver's mouth within the mouth detection window MW. When the contours of the driver's eyes and mouth are detected, the state recognizer 124 may, for example, detect the contours by fitting a curve model to a distribution of edges EG within each window; however other methods may also be used.

Next, the state recognizer 124 recognizes the eye-opening rate and mouth-opening rate of the occupant on the basis of a positional relationship of a plurality of feature points in the contours of the eyes and mouth detected by the state recognizer 124. For example, in the case of the contour of the eyes, the plurality of feature points include a first feature point, which is an end closest to the in-vehicle camera 70 in the lateral direction (corresponding to an outer corner of the eye), a second feature point, which is the upper end, and a third feature point, which is the lower end. In the case of the contour of the mouth, the end closest to the in-vehicle camera 70 in the lateral direction (corresponding to the mouth) may be replaced with the first feature point, the upper end with the second feature point, and the lower end with the third feature point. Below, as an example, a method for recognizing the eye-opening rate of the right eye in the eye detection window EW1 will be described.

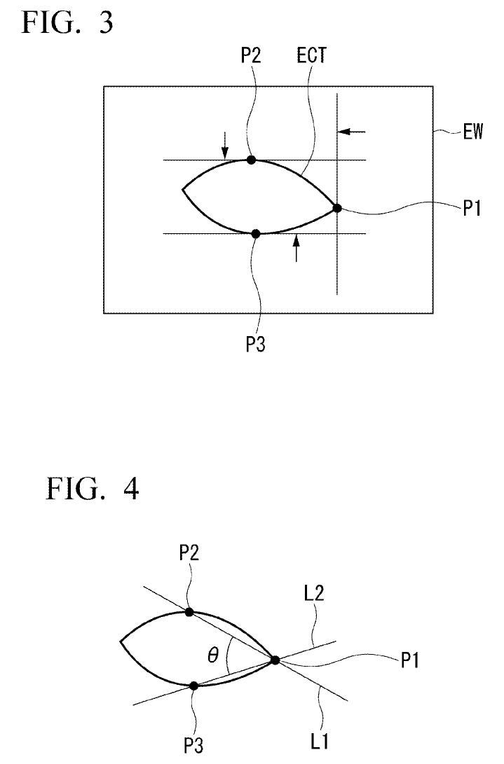

FIG. 3 is a diagram (part 1) for describing an example of the method for recognizing an eye-opening rate. FIG. 4 is a diagram (part 2) for describing an example of the method for recognizing an eye-opening rate. In FIGS. 3 and 4, P1 indicates a first feature point, P2 indicates a second feature point, and P3 indicates a third feature point. The state recognizer 124 sets a vertical line within the eye detection window EW1, virtually moves the vertical line from a right end of the eye detection window EW1 to the left, and determines a point of intersection where the vertical line first intersects with the eye outline ECT as a first feature point P1. The state recognizer 124, for example, sets a horizontal line within the eye detection window EW1, virtually moves the set horizontal line from a top end of the eye detection window EW1 to the bottom, and determines a point of intersection where the horizontal line first intersects with the eye outline ECT as a second feature point P2. For example, the state recognizer 124 virtually moves the horizontal line from a bottom edge of the eye detection window EW1 to the top within the eye detection window EW1, and determines a point of intersection where the horizontal line first intersects with the eye outline ECT as a third feature point P3.

Then, as shown in FIG. 4, the state recognizer 124 acquires an angle θ between a first line L1 connecting the first feature point P1 and the second feature point P2, and a second line L2 connecting the first feature point P1 and the third feature point P3, and recognizes an eye-opening rate α of the driver on the basis of the acquired angle θ. For example, the state recognizer 124 defines a reference angle θ₀, which is an average of the angle θ acquired on the basis of captured images taken over a first few minutes after the driver gets into the vehicle M, as a state where the eye-opening rate α is 100%. The state recognizer 124 then divides any subsequently acquired angle θ by the reference angle θ₀ and multiplies a result value of the division by 100 to recognize the eye-opening rate α while driving (for example, eye-opening rate α=(θ/θ₀)×100).

The reference angle θ₀ may be acquired in advance and stored in the storage 190. When the eye-opening rate α is recognized, the state recognizer 124 may read the reference angle θ₀ from the storage 190 and use it in the calculation. The reference angle θ₀ may be a preset fixed value, or it may be adjusted as needed to match the average angle while the driver is driving obtained from a time series of captured images.

The state recognizer 124 also uses the method described above to recognize the eye-opening rate of a left eye and mouth-opening rate of a mouth of the driver. The state recognizer 124 may also recognize an eye closing rate and a mouth closing rate instead of the eye-opening rate and mouth-opening rate. In this case, for example, in a case of the eyes, the state recognizer 124 recognizes a value obtained by subtracting the eye-opening rate α from 1 as the eye-closing rate, and a value obtained by subtracting the opening rate from 1 as the mouth-closing rate.

The state recognizer 124 may also recognize a line of sight (a direction in which the driver is looking) of the driver of the vehicle M or a direction of the driver’s face on the basis of results of image analysis processing of the images captured by the in-vehicle camera 70. For example, the state recognizer 124 may use template matching or other methods to detect a combination of reference points (portions of the eyes that do not move) and moving points (portions of the eyes that move) of the driver's eyes from the image. Examples of the combination of reference points and moving points include a combination of an inner corner of the eye and the iris, a combination of a corneal reflection area and the pupil, and the like. The corneal reflection area is, for example, an area of a cornea that reflects infrared light when the in-vehicle camera 70 has emitted infrared light toward the driver. The state recognizer 124 then recognizes the line of sight of the driver by performing coordinate transformation from an image plane to real space on the basis of a position of a moving point relative to the reference point. The state recognizer 124 recognizes the direction of the driver's face on the basis of position information (for example, relative position information for each part) of the eyes, nose, mouth, and the like within a face area obtained from results of analyzing the image.

### [State determiner]

Next, the function of the state determiner 142 will be described in detail. The state determiner 142 determines the state of the driver on the basis of information recognized by the state recognizer 124, the behavior recognizer 126, the traveling controller 180, and the like. The state of the driver may include the first state (for example, inattentive driving) and the second state (for example, drowsy driving) described above, as well as a third state. The third state, for example, refers to distracted driving.

Here, inattentive driving refers to, for example, a state in which the driver lacks attention to a traveling direction of the vehicle M. Inattentive driving refers to a state in which the driver pays attention to a surrounding scenery, an image displayed on the navigation device 50, conversations with passengers, and the like and is unable to concentrate on driving, and is known as so-called “external inattention to the road.” Inattentive driving may also include, for example, a state in which the line of sight or facial direction of the driver is not within a predetermined monitoring range (a predetermined range including the traveling direction) for the traveling direction of the vehicle M. In a case of inattentive driving, according to results of analysis using images captured by the in-vehicle camera 70, it may be sometimes recognized that the driver has his or her eyes closed (for example, when the driver is looking down or thinking with his or her eyes closed). Drowsy driving refers to, for example, a state in which the driver has his or her eyes continuously closed. Distracted driving refers to, for example, a state in which the driver's eyes are open but do not concentrate on driving. For example, even if the line of sight or facial direction is directed toward a monitoring range of the vehicle M, the driver may be daydreaming or thinking about something other than driving, which can lead to overlooking danger, and is known as so-called “intrinsic inattention to a road ahead.”

The state determiner 142 determines that the driver has his or her eyes closed when the eye-opening rate of the driver's left and right eyes recognized by the state recognizer 124 is less than a first threshold value, and determines that the driver does not have his or her eyes closed (has the eyes open) when the rate is equal to or greater than the first threshold value. By performing determination using the eye-opening rates of both the left and right eyes, it is possible to determine more accurately whether the driver is performing drowsy driving in subsequent processing. When the eye-opening rate of only one of the eyes can be recognized from the image captured by the in-vehicle camera 70, the eye-opening rate of the eye alone may be used to determine whether the driver has his or her eyes closed. As a result, it is possible to determine whether the driver has his or her eyes closed even if the driver turns sideways and only one eye is captured. In the following description, “having eyes closed” refers to both the left and right eyes being closed. In the following description, methods for determining the state of the driver are divided into several patterns and described.

### First determination pattern

In a first determination pattern, the state determiner 142 determines that the state of the driver is the second state (for example, drowsy driving) when the driver has his or her eyes continuously closed and a duration of driving of the vehicle M by the driver, recognized by the behavior recognizer 126, is equal to or greater than a first predetermined period of time. A case in which the driver has his or her eyes continuously closed is, for example, when the state determined by the state determiner 142 that the driver has his or her eyes closed continues for a second predetermined period of time or longer. In the first determination pattern, the state determiner 142 may determine that the driver is in the first state (for example, inattentive driving) or not in the second state when the duration of driving of the vehicle M by the driver is less than the first predetermined period of time. The first predetermined period of time may be adjusted depending on the speed of the vehicle M and the surrounding conditions (for example, an inter-vehicle distance from a preceding vehicle). In this case, as the speed becomes higher or the inter-vehicle distance becomes larger, the first predetermined period of time becomes shorter.

### Second determination pattern

In a second determination pattern, the state determiner 142 determines that the state of the driver is the second state when the driver has his or her eyes continuously closed and the driving mileage recognized by the behavior recognizer 126 is equal to or greater than a first predetermined distance. When the driving mileage is less than the first predetermined distance, the state determiner 142 determines that the driver is in the first state or not in the second state.

### Third determination pattern

In a third determination pattern, the state determiner 142 determines that the state of the driver is the first state when the driver has his or her eyes continuously closed and when a speed at which the driver closes his or her eyes (eye closing speed) is equal to or greater than a first predetermined speed. It also determines that the state of the driver is the second state when the eye closing speed is less than the first predetermined speed. The eye closing speed may be derived on the basis of, for example, an amount of change in the eye-opening rate over a third predetermined period of time, or derived on the basis of an amount of change in distance (difference distance) between the second feature point P2 and the third feature point P3 shown in FIGS. 3 and 4 over the third predetermined period of time. The difference distance is a difference (d1-d2) between a distance d1 between the second feature point P2 and the third feature point P3 at a certain time and a distance d2 between the second feature point P2 and the third feature point P3 at a time after a third predetermined period of time elapses from that time. The third predetermined period of time is, for example, a short time equal to or shorter than a time it takes to change from an eye-open state to an eye-closed state. The first predetermined speed may be a fixed speed based on an average eye-closing speed when drowsy driving generally starts, or may be a speed based on an eye-closing speed when blinking while each driver performs driving. As a result, the state of the driver can be more appropriately determined using the eye-closing speed.

### Fourth determination pattern

In a fourth determination pattern, the state determiner 142 determines that the state of the driver is the first state (or not the second state) when the driver has his or her eyes continuously closed, the eye closing speed is equal to or greater than a first predetermined speed, and the driver's mouth is closed. A case where the mouth is closed refers to, for example, when the mouth-opening rate of the driver, recognized by the state recognizer 124, is less than a second threshold value. The state determiner 142 determines that the state of the driver is the second state (or not the first state) when the driver has his or her eyes continuously closed, the eye closing speed is less than the first predetermined speed, and the driver's mouth is open. A case where the mouth is open refers to when the mouth-opening rate of the driver, recognized by the state recognizer 124, is equal to or greater than a third threshold value. The third threshold value may be the same value as the second threshold value. The second and third threshold values may be fixed values or may be variable values that correspond to a position and a shape of each driver's mouth.

### Fifth determination pattern

In a fifth determination pattern, the state determiner 142 determines that the state of the driver is the second state (or not the first state) when the driver has his or her eyes continuously closed and the behavior recognizer 126 recognizes that a behavior of the vehicle M is not stable. As a result, the state of the driver can be more appropriately determined using not only image information but also behavior information of the vehicle M.

### Sixth determination pattern

In a sixth determination pattern, the state determiner 142 determines that the state of the driver is the first state (or not the second state) when the driver has his or her eyes continuously closed, the duration of driving the vehicle M is less than the first predetermined period of time, a drowsiness level of the driver is less than a predetermined value, and a behavior of the vehicle M is stable. The drowsiness level of the driver is an index value indicating a degree of drowsiness, and the value increases as the drowsiness level becomes higher, for example. The drowsiness level is estimated, for example, based on a manner in which the eyes are closed, and the drowsiness level has a higher value as the eye closing speed becomes slower. The state determiner 142 may acquire a drowsiness level that has a higher value as the number of eye blinks during a fourth predetermined period of time obtained from the image captured by the in-vehicle camera 70 becomes fewer. Furthermore, the drowsiness level may be estimated based on movement of the driver's mouth. In this case, the state determiner 142 acquires a drowsiness level having a higher value as the driver yawns during a fifth predetermined period of time obtained from the images captured by the in-vehicle camera 70 more frequently, for example.

### Seventh determination pattern

In a seventh determination pattern, the state determiner 142 determines that the state of the driver is the second state when the driver has his or her eyes continuously closed and a voice input from the microphone 36 of the HMI 30 contains a specific sound. The specific sound is, for example, a sound of breathing or snoring. As a result, it is possible to more accurately determine that the driver is in the second state (drowsy driving) using not only image information but also voice information. When the images captured by the in-vehicle camera 70 determine that an occupant (passenger) other than the driver is present in the vehicle M, the state determiner 142 may not perform determination processing using the seventh determination pattern. As a result, it is possible to suppress the state of the driver from being determined based on vocal sounds (such as snoring) emitted by passengers, and therefore, the state of the driver can be determined more accurately.

### Eighth determination pattern

In an eighth determination pattern, the state determiner 142 determines that the state of the driver is the second state when the driver has his or her eyes continuously closed and an elapsed time during which driving control by the traveling controller 180 is switched to manual driving by the driver is less than a sixth predetermined period of time, and determines that the state of the driver is the first state when the elapsed time is equal to or greater than the sixth predetermined period of time. For example, when the driver is performing drowsy driving while the driving control of the vehicle M (for example, fully automated driving) is being executed and then the driving control is switched to manual driving, the driver may not have fully woken up from drowsiness, and continuous eye closure thereafter is likely to indicate drowsy driving. Therefore, by including not only images but also information on a status and history of the driving control of the vehicle M by the traveling controller 180, the state of the driver can be more accurately determined.

### Ninth determination pattern

The state determiner 142 determines that the state of the driver is the third state (for example, distracted driving) when the driver does not have his or her eyes continuously closed (has the eyes open) and the behavior recognizer 126 recognizes that the behavior of the vehicle M is not stable. The state determiner 142 may also determine that the state of the driver is the third state when the driver does not have his or her eyes continuously closed and the vehicle M has deviated from the traveling lane for a seventh predetermined period of time or longer. The state determiner 142 may also determine that the state of the driver is the third state when the driver does not have his or her eyes continuously closed and the line of sight of the driver remains unchanged for an eighth predetermined period of time or longer. The seventh and eighth predetermined period of times may be fixed or may be variable depending on the driver and traveling conditions.

Each of the first through ninth determination patterns described above may be combined with at least some or a plurality of the other determination patterns, and some pieces of processing may be replaced with processing of another determination pattern. When a

plurality of determination patterns are combined, the state determiner 142 may determine the state of the driver when determination conditions of at least one of the plurality of determination patterns are met, or may determine the state of the driver when all of the determination conditions are met. In the embodiment, the state determiner 142 may select a determination pattern depending on a status of the vehicle M. In this case, a preset determination pattern is set depending on, for example, a time of day (daytime, nighttime, or the like) when the driver is driving, a surrounding weather, a presence or absence of a passenger, road conditions, and the like. Additionally, the state determiner 142 may assign priorities to the first through ninth determination patterns in advance and determine the state of the driver using a determination pattern with the highest executable priority. The state determiner 142 may select the number and type of determination patterns to combine on the basis of an age of the driver (or whether the driver is elderly), the number of times the driver has previously performed drowsy driving, inattentive driving, or distracted driving, and the like. As a result, the state of the driver can be determined using a more appropriate determination method depending on the driver.

### [Notification content]

Next, notification content corresponding to the state of the driver will be described. For example, when the state determiner 142 determines that the state of the driver is the first state (for example, inattentive driving), the HMI controller 160 generates inattentive driving notification information (first notification information) and causes the HMI 30 to output the generated inattentive driving notification information (an example of the first notification). The inattentive driving notification information may be, for example, information indicating that the driver has been determined to perform inattentive driving, or information urging the driver not to perform inattentive driving (or to pay close attention to the surroundings). The inattentive driving notification information includes at least one of an image and a voice (a warning sound).

When the state determiner 142 determines that the state of the driver is the second state (for example, drowsy driving), the HMI controller 160 generates drowsiness notification information (second notification information) that differs from the inattentive driving notification information and causes the HMI 30 to output the generated drowsy driving notification information (an example of a second notification). The drowsy driving notification information may be, for example, information indicating that the driver has been determined to perform drowsy driving, or information urging the driver not to perform drowsy driving (or to wake up). The drowsy driving notification information includes at least one of an image and a voice (a warning sound). The drowsy driving notification information may be information with a greater level of notification (warning) than the inattentive driving notification information. In this case, for example, when the inattentive driving notification information is either an image or a voice, the HMI controller 160 includes both images and audio in the drowsy driving notification information. The HMI controller 160 may increase an output voice volume of the drowsy driving notification information compared to an output voice volume of the inattentive driving notification information, adjust the voice of the drowsy driving notification information to be more noticeable to the driver, or highlight and display an image of the drowsy driving notification information using a color or text that is more noticeable to the driver than the inattentive driving notification information. As a result, it is easier for the driver to recognize an importance of the drowsy driving notification information.

After the HMI 30 outputs the drowsy driving notification information, the HMI controller 160 may generate rest prompting information that urges the driver to take a break and causes the HMI 30 to output the generated rest prompting information. The rest prompting information includes at least one of an image and a voice. In addition to the information urging to take a rest, the rest prompting information may include driving information such as the duration of driving and driving mileage, and may also include information on nearby rest spots acquired from the map information 192 on the basis of the position information of the vehicle M. The rest prompting information may also be a strong warning commanding a rest. Since continuing to drive while the driver performs drowsy driving is inappropriate, the rest prompting information can urge the driver to take a rest immediately.

When the state determiner 142 determines that the state of the driver is the third state (for example, distracted driving), the HMI controller 160 generates distracted driving notification information (third notification information) that is different from the inattentive driving notification information and the drowsiness notification information, and causes the HMI 30 to output the generated distracted driving notification information (an example of a third notification). The distracted driving notification information may be information indicating that the driver has been determined to perform distracted driving, or it may be information urging the driver not to perform distracted driving. The distracted driving notification information is information that at least has a less degree of notification than the drowsy driving notification information. The first, second, and third notifications described above are all different notifications.

The traveling controller 180 may execute predetermined driving control when the state of the driver continues (there is no change in the state) after the HMI controller 160 outputs the inattentive driving notification information, drowsy driving notification information (rest prompting information), or distracted driving notification information. For example, when the state of the driver remains in the second state even after a ninth predetermined period of time has elapsed since the HMI controller 160 output the drowsy driving notification information, the traveling controller 180 executes driving control to move the vehicle M to a safe position and stop the vehicle. When the state of the driver remains in the first or third state even after a tenth predetermined period of time has elapsed since the HMI controller 160 output the inattentive driving notification information or distracted driving notification information, the traveling controller 180 may perform driving control to move the vehicle M to a safe position and stop the vehicle, or may predict that inattentive driving of the driver will be improved in the near future and execute driving control such as ACC or LKAS. The ninth predetermined period of time described above is shorter than the tenth predetermined period of time. In this manner, by performing traveling control on the basis of the state of the driver after the notification information is notified, more appropriate driving control can be executed depending on the state of the driver.

### [Processing flow]

In the following description, processing executed by the driving assistance device 100 of the present embodiment will be described. Processing including determining the state of the driver and issuing a notification based on that state among the processing executed by the driving assistance device 100 will be mainly described in the following description.

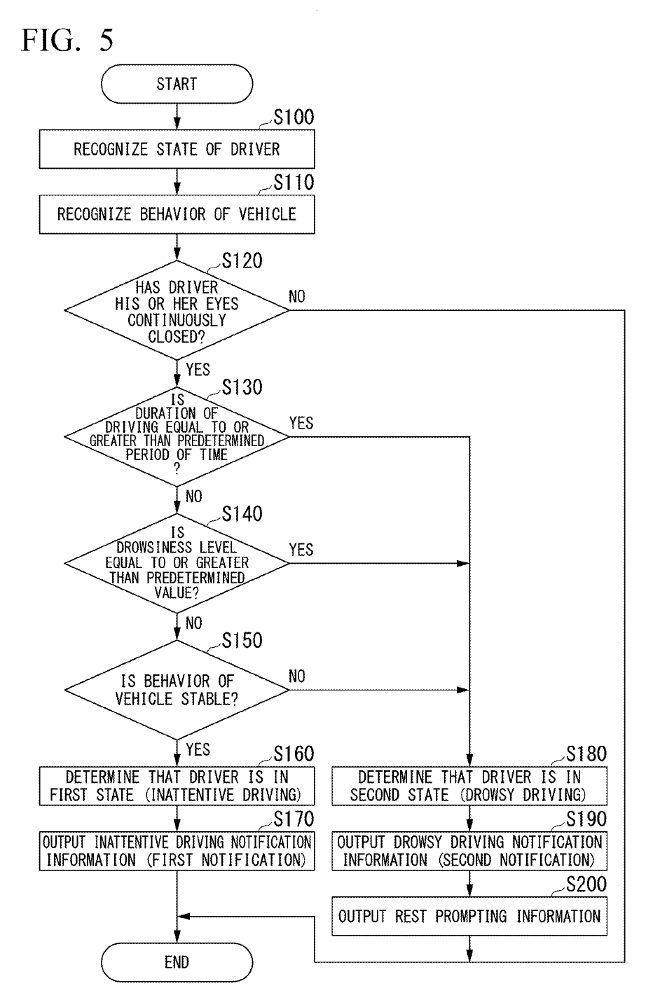

FIG. 5 is a flowchart which shows an example of the processing executed by the driving assistance device 100 in the present embodiment. In the example of FIG. 5, the state recognizer 124 recognizes the state of the driver (step S100). Next, the behavior recognizer 126 recognizes the behavior of the vehicle M (step S110). Next, the state determiner 142 determines whether the driver has his or her eyes continuously closed (step S120). When it is determined that the driver has his or her eyes continuously closed, the state determiner 142 determines whether the duration of driving of the driver is equal to or greater than a predetermined period of time (the first predetermined period of time) (step S130). When it is determined that the duration of driving is not equal to or greater than the predetermined period of time, the state determiner 142 determines whether the drowsiness level of the driver is equal to or greater than a predetermined value (step S140). When it is determined that the drowsiness level is not equal to or greater than the predetermined value, the state determiner 142 determines whether the behavior of the vehicle M is stable. When it is determined that the behavior of the vehicle M is stable, the state determiner 142 determines that the state of the driver is the first information (inattentive driving) (step S160). Next, the HMI controller 160 causes the HMI 30 to output inattentive driving notification information corresponding to the first state (performs the first notification) (step S170).

When it is determined in step S130 that the duration of driving of the driver is equal to or greater than the predetermined period of time, when it is determined in step S140 that the drowsiness level is equal to or greater than the predetermined value, or when it is determined in step S150 that the behavior of the vehicle M is not stable, the HMI controller 160 determines that the state of the driver is the second state (drowsy driving) (step S180). Next, the HMI controller 160 causes the HMI 30 to output drowsy driving notification information corresponding to the second state (performs a second notification) (step S190). Next, the HMI controller 160 causes the HMI 30 to output rest prompting information (step S200). As a result, the processing of this flowchart ends.

When it is determined in the processing of step S120 that the driver does not have his or her eyes continuously closed, processing of this flowchart ends.

The processing shown in FIG. 5 is merely an example of an embodiment, and shows processing based on the sixth determination pattern among the first through ninth determination patterns described above. However, processing based on other determination patterns may also be performed. Each determination pattern may be combined with parts of other determination patterns, or some pieces of processing may be replaced with processing of other determination patterns. For example, when it is determined in step S120 that the driver does not have his or her eyes continuously closed (has the eyes open), the state determiner 142 may perform a determination using the ninth determination pattern described above and determine whether the state of the driver is distracted driving on the basis of a result of the determination. When it is determined that the state of the driver is distracted driving, the HMI controller 160 may generate distracted driving notification information and output it to the HMI 30. In processing of step S130, the second determination pattern may be applied to determine whether the driving mileage is equal to or greater than the first predetermined distance. The processing of FIG. 5 may not include at least one of the processing of step S140 and step S150.

According to the embodiment described above, the driver state determination device includes a determiner 140 that determines the state of the driver of the vehicle M (an example of a mobile object), and a notifier (HMI controller 160, HMI 30) that notifies the driver on the basis of a result of determination by the determiner. The notifier issues the first notification when the determiner 140 determines that the state of the driver is the first state, and issues a second notification different from the first notification when the state of the occupant is a second state different from the first state. This makes it possible to more appropriately determine the state of the driver.

For example, according to the present embodiment, even if the driver has his or her eyes continuously closed, drowsiness and inattentive driving can be distinguished, and different notifications (warnings) can be output accordingly. According to the present embodiment, when the driver performs drowsy driving, a warning is issued, followed by a notification urging the driver to take a break, thereby suppressing the driver from continuing to drive during drowsy driving and allowing the driver to take a break earlier. According to the present embodiment, on the basis of the first through ninth determination patterns described above, a driving state of the driver, such as inattentive driving, drowsy driving, or distracted driving, can be more accurately determined according to not only images but also voices, the vehicle behavior, the execution status of driving control, and the like.

The embodiment described above can be expressed as follows.

A driver state determination device includes a storage medium for storing computer-readable instructions, and a processor connected to the storage medium, in which the processor executes the computer-readable instructions to determine a state of a driver of a mobile object, issue a notification to the driver on the basis of a result of the determination, issue a first notification when the state of the driver is determined to be a first state, and issue a second notification different from the first notification when the state of the driver is determined to be a second state different from the first state.

The above describes a form for implementing the present invention using an embodiment, but the present invention is not limited to such an embodiment, and various modifications and substitutions can be made within a range not departing from the gist of the present invention

What is claimed is:

1. A driver state determination device comprising:
a determiner configured to determine a state of a driver of a mobile object; and
a notifier configured to issue a notification to the driver on the basis of a determination result of the determiner,
wherein the notifier issues a first notification when the determiner determines that a state of the driver is a first state, and issues a second notification different from the first notification when the determiner determines that the state of the driver is a second state different from the first state.

2. The driver state determination device according to claim 1, wherein the first state is inattentive driving and the second state is drowsy driving.

3. The driver state determination device according to claim 2, wherein, when the determiner determines that the state of the driver is the second state, the notifier issues a notification corresponding to the drowsy driving, and then issues a notification urging the driver to take a rest.

4. The driver state determination device according to claim 1,
wherein, when the driver has his or her eyes continuously closed and the driver has been driving the mobile object for a predetermined period of time or longer, the determiner determines that the state of the driver is the second state.

5. The driver state determination device according to claim 1,
wherein the determiner determines that the state of the driver is the first state when the driver has his or her eyes continuously closed and a speed at which the driver closes his or her eyes is equal to or greater than a predetermined speed, and determines that the state of the driver is the second state when the speed at which the driver closes his or her eyes is less than the predetermined speed.

6. The driver state determination device according to claim 1,
wherein the determiner determines that the state of the driver is the first state when the driver has his or her eyes continuously closed and the speed at which the driver closes his or her eyes is equal to or greater than a predetermined speed and the driver's mouth is closed, and determines that the state of the driver is the second state when the speed at which the driver closes his or her eyes is less than the predetermined speed and the driver's mouth is open.

7. The driver state determination device according to claim 1,
wherein the determiner determines that the state of the driver is the second state when the driver has his or her eyes continuously closed and a behavior of the mobile object is not stable.

8. The driver state determination device of claim 1,
wherein the determiner determines that the state of the driver is the first state when the driver has continuously closed his or her eyes, a duration of driving the mobile object is less than a predetermined period of time, a drowsiness level of the driver is less than a predetermined value, and the behavior of the mobile object is determined to be stable.

9. A driver state determination method comprising:
by a computer,
determining a state of a driver of a mobile object;
issuing a notification to the driver on the basis of a result of the determination;
issuing a first notification when a state of the driver is determined to be a first state; and
issuing a second notification different from the first notification when the state of the driver is determined to be a second state different from the first state.

10. A computer-readable non-transitory storage medium that has stored a program causing a computer to execute:
determining a state of a driver of a mobile object;
issuing a notification to the driver on the basis of a result of the determination;
issuing a first notification when a state of the driver is determined to be a first state; and
issuing a second notification different from the first notification when the state of the driver is determined to be a second state different from the first state.
